(19) United States
(12) Patent Application Publication
(10) Pub. No.: US 2017/0290947 A1
(43) Pub. Date: Oct. 12, 2017

(54) MICRONIZED HYDROPHILIC CROSS-LINKED BIOPOLYMER SYSTEMS AND METHOD OF MAKING SAME

(71) Applicant: Therakine Bio Delivery GmbH, Berlin (DE)

(72) Inventors: Andreas VOIGT, Berlin (DE); Sonja LEHMANN, Berlin (DE); Mariana DOBRANIS, Berlin (DE); Annette ASSOGBA-ZANDT, Berlin (DE)

(21) Appl. No.: 15/516,685

(22) PCT Filed: Oct. 7, 2015
(86) PCT No.: PCT/US2015/054378

§ 371 Date: Apr. 4, 2017

Related U.S. Application Data
(60) Provisional application No. 62/061,201, filed on Oct. 8, 2014.

(51) Int. Cl. A61L 17/00; C08B 37/08; A61K 9/00; C08L 5/08; A61L 31/00; A61K 47/36

(52) U.S. Cl. CPC A61L 17/005; A61L 31/00; A61K 47/36; A61K 9/0019; C08L 5/08; C08B 37/0072

(57) ABSTRACT

Disclosed are micronized hydrophilic systems of highly concentrated, cross-linked biopolymers. The system is created by combining a biopolymer with a cross-linking agent under mechanical kneading and allowing the biopolymer to undergo a cross-linking process followed by purification, drying and milling. The resulting micronized biopolymer system has an increased biopolymer concentration and increased longevity within the body.

### CROSS-REFERENCE TO RELATED APPLICATIONS

The present application is a national stage entry according to 35 U.S.C. §371 of PCT Application No. PCT/US2015/54378 filed on Oct. 7, 2015, which claims priority to U.S. Provisional Application Ser. No. 62/061,201 filed on Oct. 8, 2014.

### BACKGROUND

### Technical Field

The subject matter herein generally relates to micronized hydrophilic biopolymer systems. More specifically, the subject matter relates to dehydrated micronized hydrophilic biopolymer systems and methods for manufacturing the same.

### Background

Traditional biopolymer systems are dissolved and cross-linked to provide systems from free solution to gel consistency. Thereafter, they are washed, dried and milled. Such systems have insufficient biopolymer concentration and suffer significant swelling after resuspension. Therefore, there is an ongoing need in the field for an improved microscopic biopolymer system with increased biopolymer concentration and segment density.

Specifically, there is a significant unmet need for a biopolymer system that has limited volumetric increase of the cross-linked biopolymer system after resuspension and has a long life within human or animal body.

### SUMMARY

Various embodiments are described here, and do not limit the scope in any way.

In a non-limiting embodiment, a method of manufacturing a biopolymer system is disclosed. In another embodiment, this method comprises mixing at least one polymer with at least one cross-linker by pressing and folding in repeated cycles in an algorithmic manner, where the mixing produces a partly cross-linked biopolymer, purifying the partly cross-linked biopolymer, transferring the partly cross-linked biopolymer to an aqueous solution comprising at least one cross-linker for second cross-linking, the second cross-linking forming a macroscopic biopolymer system, purifying, drying and partly dehydrating the macroscopic biopolymer system, and micronizing the dried macroscopic biopolymer system to a predetermined average particle diameter.

In yet another embodiment, the polymer is selected from the group consisting of hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein and whey protein. In still yet another embodiment, the cross-linker is selected from the group consisting of 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4-[N-maleimidomethyl]cyclohexane-1-carboxylate) (SMCC), and (sulfosuccinimidyl 4[N-maleimidomethyl]cyclohexane-1-carboxylate) (Sulfo-SMCC). In another embodiment, the physicochemical properties of each biopolymer are maintained throughout the process. In yet another embodiment, the partly cross-linked biopolymer is in a highly concentrated and a low level hydrated form. In still yet another embodiment, the biopolymer system is a translucent, gelatinous composition with an increased concentration of the polymer or a translucent, liquid composition with an increased concentration of the polymer. In another embodiment, the macroscopic biopolymer is micronized by mechanical milling and sieving. In yet another embodiment, the biopolymer comprises a system of increased polymer concentration and segment density.

In another embodiment, there is a delivery system, comprising the biopolymer system manufactured according to method described above. In yet another embodiment, the biopolymer system delivers materials selected from the group consisting of biologics, biomaterials, pharmaceutically active compounds, cosmetics, food and food additives. In still yet another embodiment, the pharmaceutically active compound is selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In still yet another embodiment, the pharmaceutically active compound is either alone or in combination with at least one excipient. In yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In yet another embodiment, the delivery system is an injectable system comprising said biopolymer system suspended in a macroscopic cross-linked gel.

In another embodiment, there is a surgical kit, comprising the biopolymer system manufactured according to the above-mentioned method. In yet another embodiment, there is a biocompatible scaffold, comprising the biopolymer system manufactured according to the method described above. In still yet another embodiment, the scaffold is used in surgery, to plug the blood circulation system, or a combination thereof.

In another non-limiting embodiment, there is a method of manufacturing a biopolymer system described. In another embodiment, this method comprises mixing at least one polymer with at least one cross-linker by pressing and folding in repeated cycles in an algorithmic manner, where the mixing produces a partly cross-linked biopolymer, incubating and dissolving the partly cross-linked biopolymer in an aqueous solution, conducting a second cross-linking by combining the dissolved partly cross-linked biopolymer with a previously cross-linked, hydrated biopolymer, the second cross-linking forming a macroscopic biopolymer system, purifying, drying and partly dehydrating the macroscopic biopolymer system, and micronizing the dried macroscopic biopolymer system to a predetermined average particle diameter.

In yet another embodiment, the polymer is selected from the group consisting of hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein and whey protein. In still yet another embodiment, the cross-linker is selected from the group consisting of 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4-[N-maleimidomethyl]cyclohexane-1-carboxylate) (SMCC), and (sulfosuccinimidyl 4[N-maleimidomethyl]cyclohexane-1-carboxylate) (Sulfo-SMCC). In another embodiment, the physicochemical properties of each biopolymer are maintained throughout the process. In yet another embodiment, the partly cross-linked biopolymer is in a highly concentrated and a low level hydrated form. In still yet another embodiment, the macroscopic biopolymer is micronized by mechanical milling and sieving. In another embodiment, the biopolymer comprises a system of increased polymer concentration and segment density.

In another embodiment, there is a delivery system, comprising the biopolymer system manufactured according to the method described above. In yet another embodiment, the biopolymer system delivers materials selected from the group consisting of biologics, biomaterials, pharmaceutically active compounds, cosmetics, food and food additives. In still yet another embodiment, the pharmaceutically active compound is selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In another embodiment, the pharmaceutically active compound is either alone or in combination with at least one excipient. In yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In still yet another embodiment, the delivery system is an injectable system comprising said biopolymer system suspended in a macroscopic cross-linked gel. In another embodiment, there is a surgical kit, comprising the biopolymer system manufactured according to method described above. In yet another embodiment, there is a biocompatible scaffold, comprising the biopolymer system manufactured according to the above-mentioned method. In still yet another embodiment, the scaffold is used in surgery, to plug the blood circulation system, or a combination thereof.

In another non-limiting embodiment, a method of manufacturing a biopolymer system is disclosed. In another embodiment, this method comprises mixing at least one polymer with at least one cross-linker by pressing and folding in repeated cycles in an algorithmic manner, where the mixing produces a partly cross-linked biopolymer, washing and drying the partly cross-linked biopolymer, micronizing the washed and dried partly cross-linked biopolymer, incubating the micronized partly cross-linked biopolymer with an aqueous solution comprising the biopolymer system, another biopolymer system or a mixture of biopolymer system to form a macroscopic biopolymer system, purifying the macroscopic biopolymer system, and storing the macroscopic biopolymer system. In another embodiment, the polymer is selected from the group consisting of hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein and whey protein. In yet another embodiment, the cross-linker is selected from the group consisting of 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4-[N-maleimidomethyl]cyclohexane-1-carboxylate) (SMCC), and (sulfosuccinimidyl 4[N-maleimidomethyl]cyclohexane-1-carboxylate) (Sulfo-SMCC).

In still yet another embodiment, the physicochemical properties of each biopolymer are maintained throughout the process. In another embodiment, the partly cross-linked biopolymer is in a highly concentrated and a low level hydrated form. In yet another embodiment, the macroscopic biopolymer is micronized by mechanical milling and sieving. In still yet another embodiment, the biopolymer comprises a system of increased polymer concentration and segment density. In another embodiment, there is a delivery system, comprising the biopolymer system manufactured according to method described above. In yet another embodiment, the biopolymer system delivers materials selected from the group consisting of biologics, biomaterials, pharmaceutically active compounds, cosmetics, food and food additives. In still yet another embodiment, the pharmaceutically active compound is selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In another embodiment, the pharmaceutically active compound is either alone or in combination with at least one excipient. In yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In yet another embodiment, the delivery system is an injectable system comprising said biopolymer system suspended in a macroscopic cross-linked gel. In another embodiment, there is a surgical kit, comprising the biopolymer system manufactured according to the method described above. In another embodiment, there is a biocompatible scaffold, comprising the biopolymer system manufactured according to method described above. In yet another embodiment, the scaffold is used in surgery, to plug the blood circulation system, or a combination thereof.

In another non-limiting embodiment, a method of manufacturing a biopolymer system is disclosed. In another embodiment, this method comprises mixing at least one polymer with at least one cross-linker by pressing and folding in repeated cycles in an algorithmic manner, where the mixing produces a partly cross-linked biopolymer, where the partly cross-linked biopolymer is in a microscopic or macroscopic form, conducting second cross-linking by incubating and dissolving the partly cross-linked biopolymer in an aqueous solution comprising a biopolymer, where the second cross-linking forms a macroscopic biopolymer system, and purifying and storing the macroscopic biopolymer system. In yet another embodiment, the polymer is selected from the group consisting of hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein and whey protein. In still yet another embodiment, the cross-linker is selected from the group consisting of 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4[N-maleimidomethyl]cyclohexane-1-carboxylate) (SMCC), and (sulfosuccinimidyl 4-[N-maleimidomethyl]cyclohexane-1-carboxylate) (Sulfo-SMCC).

In another embodiment, the physicochemical properties of each biopolymer are maintained throughout the process. In yet another embodiment, the partly cross-linked biopolymer is in a highly concentrated and a low level hydrated form. In still yet another embodiment, the biopolymer system is a translucent, gelatinous composition with an increased concentration of the polymer or a translucent, liquid composition with an increased concentration of the polymer. In another embodiment, the macroscopic biopolymer is micronized by mechanical milling and sieving. In yet another embodiment, the biopolymer comprises a system of increased polymer concentration and segment density. In another embodiment, there is a delivery system, comprising the biopolymer system manufactured according to the method described above. In yet another embodiment, the biopolymer system delivers materials selected from the group consisting of biologics, biomaterials, pharmaceutically active compounds, cosmetics, food and food additives. In still yet another embodiment, the pharmaceutically active compound is selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In another embodiment, the pharmaceutically active compound is either alone or in combination with at least one excipient. In yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In still yet another embodiment, the delivery system is an injectable system comprising said biopolymer system suspended in a macroscopic cross-linked gel. In another embodiment, there is a surgical kit, comprising the biopolymer system manufactured according to method described above. In yet another embodiment, there is a biocompatible scaffold, comprising the biopolymer system manufactured according to method described above. In still yet another embodiment, the scaffold is used in surgery, to plug the blood circulation system, or a combination thereof.

In another non-limiting embodiment, a method of manufacturing a biopolymer system is disclosed. In another embodiment, this method comprises mixing at least one polymer with at least one cross-linker by pressing and folding in repeated cycles in an algorithmic manner, where the mixing produces a partly cross-linked biopolymer, purifying and drying the partly cross-linked biopolymer, and micronizing the dried partly cross-linked biopolymer system to a predetermined average particle diameter. In another embodiment, the polymer is selected from the group consisting of hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein and whey protein. In yet another embodiment, the cross-linker is selected from the group consisting of 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4-[N-maleimidomethyl]cyclohexane-1-carboxylate) (SMCC), and (sulfosuccinimidyl 4[N-maleimidomethyl]cyclohexane-1-carboxylate) (Sulfo-SMCC).

In another embodiment, the physicochemical properties of each biopolymer are maintained throughout the process. In yet another embodiment, the partly cross-linked biopolymer is in a highly concentrated and a low level hydrated form. In still yet another embodiment, the biopolymer system is a translucent, gelatinous composition with an increased concentration of the polymer or a translucent, liquid composition with an increased concentration of the polymer. In another embodiment, the biopolymer system is micronized by mechanical milling and sieving. In yet another embodiment, the biopolymer comprises a system of increased polymer concentration and segment density. In another embodiment, there is a delivery system, comprising the biopolymer system manufactured according to the method described above. In yet another embodiment, the biopolymer system delivers materials selected from the group consisting of biologics, biomaterials, pharmaceutically active compounds, cosmetics, food and food additives. In still yet another embodiment, the pharmaceutically active compound is selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In another embodiment, the pharmaceutically active compound is either alone or in combination with at least one excipient. In yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In still yet another embodiment, the delivery system is an injectable system comprising said biopolymer system suspended in a macroscopic cross-linked gel. In another embodiment, there is a surgical kit, comprising the biopolymer system manufactured according to the method described above. In yet another embodiment, there is a biocompatible scaffold, comprising the biopolymer system manufactured according to the method described above. In still yet another embodiment, the scaffold is used in surgery, to plug the blood circulation system, or a combination thereof.

Those skilled in the art will recognize additional features and advantages upon reading the following detailed description, and upon viewing the accompanying drawings.

### BRIEF DESCRIPTION OF THE DRAWINGS

Implementations of the present technology will now be described, by way of example only, with reference to the attached figures.

FIG. 1 shows the summary overview of the exemplary process described in Example 1.

FIG. 2 shows the summary overview of the exemplary process described in Example 2.

### DETAILED DESCRIPTION

The following language and descriptions of various embodiments are set forth in order to provide a thorough understanding of the embodiments described herein. However, it will be understood by those of ordinary skill in the art that no limitations are intended, and that further alterations, modifications, and applications of the principles of the present non-limiting are also included.

As used herein, the term “kneaded” is defined as comprising performing repeated cycles of pressing and folding, in an algorithmic manner.

As used herein, the term “micronized” is defined as having been through the process of reducing the average diameter of a solid material's particles.

As used herein, the term “cross-linking” is defined as the process of chemically joining two or more polymer chains together.

As used herein, the term “cross-linker” is defined as a reagent containing two or more reactive ends that are capable of attaching to specific functional groups on proteins or other molecules.

As used herein, the term “hydration” comprises water contained within a crystalline structure of the biopolymer structure and water bound to or within a biopolymer composition.

As used herein, the term “dried” is defined as essentially free of water other than water contained within a crystalline structure of the biopolymer structure.

As used herein, the term “pharmaceutically active compound” refers to a compound or a combination of compounds that are used in manufacturing a drug product. This compound may also have a direct effect on the disease diagnosis, prevention, treatment or cure. Some examples of the pharmaceutically active compound that can be used herein are listed supra.

As used herein, the term “receptor antagonist” refers to a type receptor specific ligand or drug that can block receptor-mediated response by binding to the receptor and preventing the binding of agonists to the receptor. Some examples of such receptor antagonist include but are not limited to anti-TNF alpha, anti-Interleukin-1, anti-Interleukin-6, anti-epidermal growth factor receptor, anti-dopamine receptor, anti-Angiotensin II receptor, anti-aldosterone receptor and anti-leukotriene receptor.

As used herein, the term “anti-angiogenic compounds” refer to compounds that inhibit the growth of new blood vessels, reduce the production of pro-angiogenic factors, prevent the pro-angiogenic factors from binding to their receptors, and block the actions of pro-angiogenic factors or a combination thereof. Some examples of these compounds include but are not limited to compounds that inhibit the activity of VEGF, PDGF, and angiogenesis stimulators.

As used herein, the term “intracellular signaling inhibitors” refer to compounds that block signaling pathways by blocking the binding of ligands to the receptor involved in cell signaling or signal transduction, the actions of the receptors or the combination thereof. These compounds are useful in treatment, prevention, diagnosis or cure of various diseases. Some examples of intracellular signaling inhibitors include but are not limited to JAK1, JAK3 and SYK.

As used herein, the term “excipient” is known in the art to refer to a natural or synthetic substance is formulated alongside the pharmaceutically active compound. There are several reasons for using the excipient in a drug composition because they act as a buffer, filler, binder, lubricant, or an osmotic agent. For instance, it may be used for the purpose of bulking up formulations that contain potent pharmaceutically active compounds. It may also be used to confer a therapeutic enhancement on the pharmaceutically active compound in the final dosage form, such as facilitating drug absorption or solubility. Further, it may also be used to assist in the handling of the pharmaceutically active compound by enabling powder consistency, non-stick properties or in vitro stability such as prevention of denaturation. Some of the factors that affect the selection of the excipient in a drug composition may include but is not limited to the route of administration, dosage form as well as the type of the pharmaceutically active compound in the drug composition. The various classes and types of pharmaceutically active compounds, excipients, polymers, and polyampholytes are familiar to those skilled in the art of drug delivery.

As used herein, the term “macroscopic biopolymer system” refers to a biopolymer body that is macroscopic in all three space dimensions and is larger than 1 mm in all directions.

As used herein, the term “microscopic biopolymer system” refers to a biopolymer body that is microscopic in at least one spatial dimension, comprising microparticles and thin films. It is understood that “micro” is defined as 1×10E-6.

As used herein, the term “biocompatible scaffold” refers to any scaffold or matrix that comprises a biopolymer composition as described herein, wherein the scaffold or matrix is safe when given by any route of administration (e.g., intravitreal, topical, oral, intradermal, intraperitoneal, intramuscular, subcutaneous, intravenous, intrathecal, etc) and will not cause immune rejection.

The biopolymer composition may be any of a wide variety of agents, which are known to those skilled in the art. Suitable polymers include, but are not limited to, hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein, and whey protein. The method of manufacturing the micronized hydrophilic biopolymer system is the same for each biopolymer; however, the physicochemical properties of each biopolymer are maintained throughout the process.

The cross-linker may be any of a wide variety of agents, which are known to those skilled in the art. Suitable cross-linkers include, but are not limited to, 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4-[N-maleimidomethyl]cyclohexane-1-carboxylate) (SMCC), and (sulfosuccinimidyl 4-[N-maleimidomethyl]cyclohexane-1-carboxylate) (Sulfo-SMCC). The method of manufacturing the biopolymer system is the same for each cross-linker with respect to the mechanical preparation; however, the cross-linkers maintain their original chemical properties.

According to an exemplary embodiment, hyaluronic acid is partly cross-linked in a highly concentrated and hydrated configuration. The cross-linked configuration is transferred to an aqueous solution (for example, aqueous hyaluronic acid solution) for a second cross-linking. In at least one embodiment, the resulting macroscopic system is a translucent, gelatinous composition, having an increased concentration of hyaluronic acid. In other embodiments, the resulting macroscopic composition is a translucent, liquid composition, having an increased concentration of hyaluronic acid. The obtained system can be used as prepared or milled down to microparticles.

According to an exemplary embodiment, the cross-linked hyaluronic acid composition is a bulk phase material. As a result, the macroscopic composition comprises a heterogeneous mixture of cross-linked hyaluronic acid at a micro-scale. Throughout the composition, sites of low density, attained from dissolved hyaluronic acid crosslinking, and sites of high density, attained from cross-linked micronized hydrated hyaluronic acid, exist, creating a system with increased hyaluronic acid concentration and segment density.

According to exemplary embodiments there can be several sizes or size distributions of high concentration hyaluronic acid cross-linked microparticles contained in one system. According to an exemplary embodiment, a partly cross-linked, hydrated biopolymer macroscopic composition is purified, dried and micronized. The micronized composition is further manufactured to predetermined average particle sizes by sieving. According to an exemplary embodiment, a second cross-linking can be conducted to connect both a dissolved biopolymer and a hydrated, partly cross-linked biopolymer system. The resulting fully cross-linked, hydrated biopolymer macroscopic composition is purified, dehydrated and micronized. The micronized composition is further manufactured to predetermined average particle sizes by sieving.

According to an exemplary embodiment, the cross-linked biopolymer compositions have an increased longevity in a human or animal body. In at least one embodiment, the cross-linked biopolymer compositions created are functional for use in cosmetic surgery. In another embodiment, the cross-linked biopolymer compositions created are functional for use as delivery matrices for biologics, biomaterials, active pharmaceutical ingredients (APIs), cosmetics, and foods or food additives. In at least one embodiment, the cross-linked biopolymer compositions created are functional for use in biocompatible scaffolds. In another embodiment, the cross-linked biopolymer compositions created are functional for use in surgical scaffolds. In another embodiment, the cross-linked biopolymer particles can be used to create a plug in a blood circulation system.

In at least one embodiment, the cross-linked biopolymer compositions are in a dried and hydrated form wherein the cross-linked biopolymer compositions contain water within a crystalline structure of the biopolymer composition and water bound to or within a biopolymer composition. In at least one embodiment, the cross-linked biopolymer compositions are in a dried and hydrated form wherein the cross-linked biopolymer compositions contain water within a crystalline structure of the biopolymer composition or water bound to or within a biopolymer composition. In at least one embodiment, the cross-linked biopolymer compositions are in a dried and dehydrated form wherein the cross-linked biopolymer compositions do not contain water within a crystalline structure of the biopolymer composition or water bound to or within a biopolymer composition. In at least one embodiment, the micronized, cross-linked biopolymer compositions can be suspended in a macroscopic cross-linked gel, while maintaining an injectable system.

In the following, specific examples are described.

### EXAMPLES

Various embodiments are further illustrated by the following examples. The following examples shall not limit the scope in any way.

### Example 1

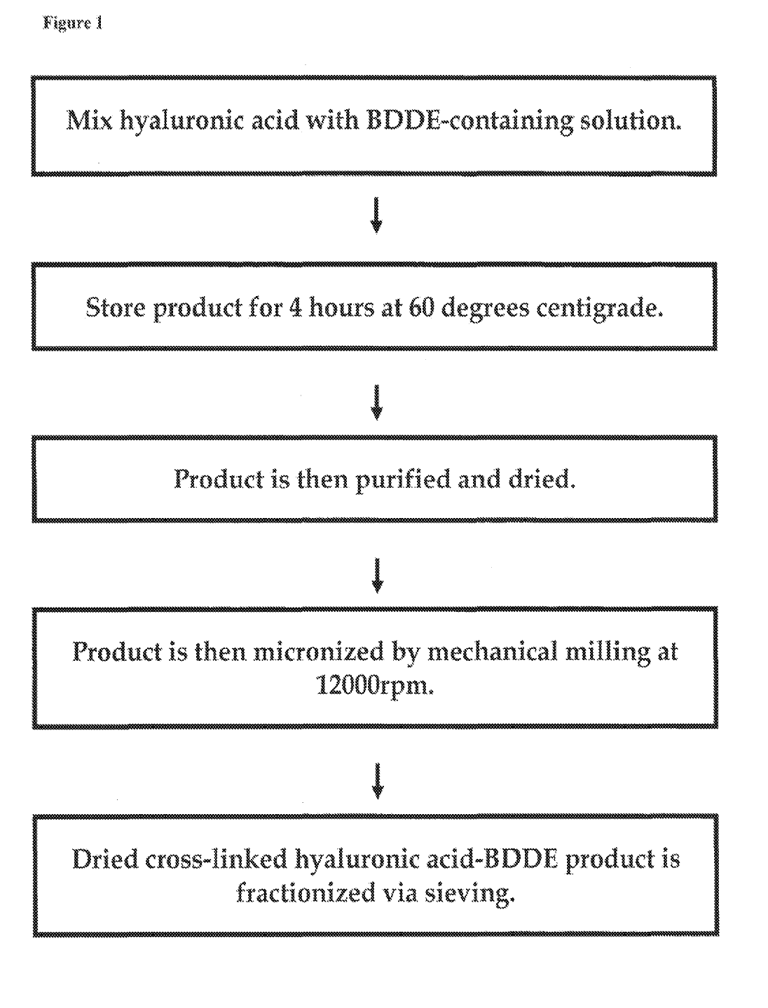

One gram of hyaluronic acid was mixed by kneading with 2 ml of a BDDE-containing solution (ratio of BDDE to glacial acetic acid: 2:1, ratio of this mixture to water: 1:4). The product was stored for 4 hours at 60 degrees centigrade. The cross-linked hyaluronic acid-BDDE product was then purified and dried. 0.5 grams of dried product was then micronized by mechanical milling at 12000 rpm (Pulverisette 14, Fritsch GmbH, Germany). After micronization, the dried cross-linked hyaluronic acid-BDDE product was fractionized via sieving. A summary overview of this exemplary process is shown in FIG. 1.

### Example 2

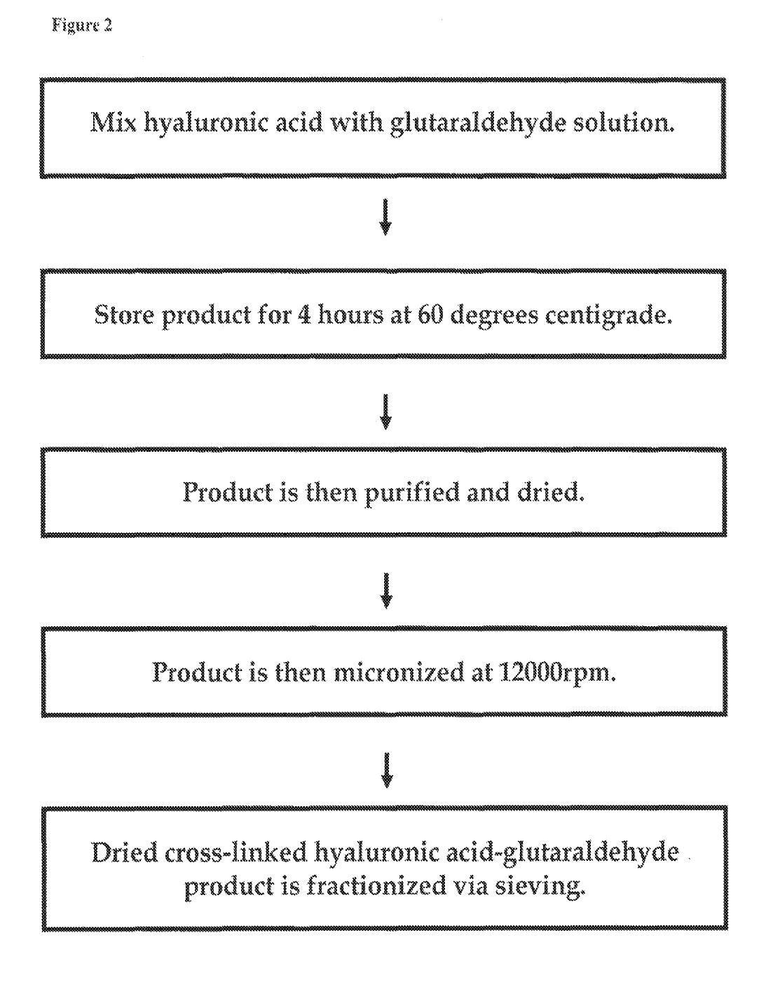

One gram of hyaluronic acid was mixed by kneading with 4 mL of a glutaraldehyde solution (99:1 (v/v %) of 50 wt % glutaraldehyde solution: 3M HCl). The product was stored for 4 hours at 60 degrees centigrade. The cross-linked hyaluronic acid-glutaraldehyde product was then purified and dried. 0.5 grams of dried product was then micronized at 12000 rpm (Pulverisette, Fritsch GmbH, Germany). After micronization, the dried cross-linked hyaluronic acid-glutaraldehyde product was fractionized via sieving. The procedure of Example 2 produced micronized and sieved cross-linked hyaluronic acid-glutaraldehyde product exhibited average particle diameters of about 50 μm to about 100 μm. A summary overview of this exemplary process is shown in FIG. 2.

While the disclosed embodiments have been particularly shown and described with reference to specific embodiments, it should be understood by those skilled in the art that various changes in form and detail may be made therein without departing from the spirit and scope of the disclosed embodiments as defined by the appended claims. The scope of the disclosed embodiments is thus indicated by the appended claims and all changes which come within the meaning and range of equivalency of the claims are therefore intended to be embraced.

What is claimed is:

1. A method of manufacturing a biopolymer system, comprising:
mixing at least one polymer with at least one cross-linker by pressing and folding in repeated cycles in an algorithmic manner, wherein said mixing produces a partly cross-linked biopolymer;
purifying said partly cross-linked biopolymer;
transferring said partly cross-linked biopolymer to an aqueous solution comprising at least one cross-linker for second cross-linking, said second cross-linking forming a macroscopic biopolymer system;
purifying, drying and partly dehydrating said macroscopic biopolymer system; and
micronizing said dried macroscopic biopolymer system to a predetermined average particle diameter.

2. The method of claim 1, wherein said polymer is selected from the group consisting of hyaluronic acid, collagen, gelatin, albumin, hemoglobin, keratin, fibrinogen, cellulose-derivatives, biogenic carbohydrates, nucleic acids, carbon hydrate, carrageenan, pectin, alginate, chitosan, casein, whey protein, and combinations thereof.

3. The method of claim 1, wherein the cross-linker is selected from the group consisting of 1,4-butanediol diglycidyl either (BDDE), dimethyl suberimidate, bissulfosuccinimidyl suberate, 1-ethyl-3-[3-dimethylaminopropyl]carbodiimide hydrochloride (EDC), glutaraldehyde, formaldehyde, (succinimidyl 4-[N-maleimidomethyl]cyclohexane-1-carboxylate) (SMCC), and (sulfosuccinimidyl 4-[N-maleimidomethyl]cyclohexane-1-carboxylate) (Sulfo-SMCC).

4. The method of claim 1, wherein the physicochemical properties of each biopolymer are maintained throughout the process.

5. The method of claim 1, wherein the partly cross-linked biopolymer is in a highly concentrated and a low level hydrated form.

6. The method of claim 1, wherein the biopolymer system is a translucent, gelatinous composition with an increased concentration of the polymer or a translucent, liquid composition with an increased concentration of the polymer.

7. The method of claim 1, wherein said macroscopic biopolymer is micronized by mechanical milling and sieving.

8. The method of claim 1, wherein said biopolymer comprises a system of increased polymer concentration and segment density.

9. A delivery system, comprising:
the biopolymer system manufactured according to claim 1.

10. The delivery system of claim 9, wherein the biopolymer system further comprises a material selected from the group consisting of biologics, biomaterials, pharmaceutically active compounds, cosmetics, food, food additives, and combinations thereof.

11. The delivery system of claim 10, wherein the pharmaceutically active compound is selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance, and a combination thereof.

12. The delivery system of claim 10, wherein the pharmaceutically active compound is either alone or in combination with at least one excipient.

13. The delivery system of claim 12, wherein the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof.

14. The delivery system of claim 9, wherein said delivery system is an injectable system comprising said biopolymer system suspended in a macroscopic cross-linked gel.

15. A surgical kit, comprising:
the biopolymer system manufactured according to claim 1.

16. A biocompatible scaffold, comprising:
the biopolymer system manufactured according to claim 1.

17. The biocompatible scaffold of claim 16, wherein said scaffold is used in surgery, to plug a blood circulation system, or a combination thereof.

18. A method of manufacturing a biopolymer system, comprising:
mixing at least one polymer with at least one cross-linker by pressing and folding in repeated cycles in an algorithmic manner, wherein said mixing produces a partly cross-linked biopolymer;
incubating and dissolving said partly cross-linked biopolymer in an aqueous solution;
conducting a second cross-linking by combining said dissolved partly cross-linked biopolymer with a previously cross-linked, hydrated biopolymer, said second cross-linking forming a macroscopic biopolymer system;
purifying, drying and partly dehydrating said macroscopic biopolymer system; and
micronizing said dried macroscopic biopolymer system to a predetermined average particle diameter.

19-83. (canceled)
